(19) United States
(12) Patent Application Publication
(10) Pub. No.: US 2025/0135061 A1
(43) Pub. Date: May 1, 2025

(54) Devices for Retaining Bodily Fluids with pH Control Components

(71) Applicant: Cupron, Inc., Richmond, VA (US)

(72) Inventors: Vikram Kanmukhla, Glen Allen, VA (US); Rachel Salvatori, Richmond, VA (US)

(73) Assignee: Cupron, Inc., Richmond, VA (US)

(21) Appl. No.: 18/681,764

(22) PCT Filed: Aug. 7, 2022
(86) PCT No.: PCT/US2022/039659

§ 371 Date: Feb. 6, 2024

Related U.S. Application Data
(60) Provisional application No. 63/230,508, filed on Aug. 6, 2021.

(51) Int. Cl. A61L 15/18; A61F 13/15; A61F 13/511; A61L 15/24; A61L 15/46

(52) U.S. Cl. CPC A61L 15/18; A61F 13/15211; A61F 13/15699; A61F 13/15731; A61F 13/15739; A61F 13/51113; A61L 15/24; A61L 15/46; A61F 2013/15235; A61L 2300/102; A61L 2300/434

(57) ABSTRACT

Devices used to absorb bodily fluids include, but are not limited to, diapers, incontinence devices, feminine hygiene products, or wound dressings, for example. Certain embodiments of these devices are produced from a top sheet, an absorbent core, and back sheet. Embodiments of the top sheet have a nonwoven polymeric sheet having both water insoluble and water soluble urease inhibiting compounds. The water insoluble and water soluble urease inhibiting compounds may be copper compounds. For example, the nonwoven polymeric top sheet have polymeric fibers and the polymeric fibers include a plurality of first particles of water insoluble copper compounds that release at least one of Cu+ ions and Cu++ ions upon contact with a body fluid embedded in the polymeric fibers and water soluble copper compounds on the surface of the polymeric fibers. The devices also include water insoluble urease inhibiting compounds within the absorbent core.

### FIELD OF THE INVENTION

Diapers, generally, comprise a top sheet, an absorbent core and a bottom sheet. Water insoluble and/or soluble copper compounds that release at least one of Cu+ ions and Cu++ ions upon contact with a fluid may be added to at least one of the top sheet, the absorbent core and the bottom sheet or another component of the diaper to inhibit the catalytic hydrolysis of urea in urine by urease enzymes, and by inhibiting microbial growth. The water insoluble or soluble copper compounds thereby reduce the rate of degradation of the urea to ammonia and carbon dioxide and, consequently, reduce the odor and pH of a used incontinence device and irritation to the skin of the wearer.

By significantly reducing the formation of ammonia, the pH of the environment within the diaper can be maintained slightly acidic and more beneficial to skin health. In an embodiment of the incontinence device, the top sheet comprises a nonwoven polymeric sheet comprising embedded water insoluble copper compounds and an absorbent core comprising water soluble copper compounds.

### DESCRIPTION

### Background

Incontinence results from the lack of control or involuntary leakage of urine or stool. Diapers, incontinence products, adult protective underwear, sanitary napkins, or other absorbent products (hereinafter “diapers”) are typically used to reduce the negative effects of an incontinence problem if therapies and medications cannot help with alleviating the incontinence itself. Diapers are used to absorb urine and retain stool to prevent leakage. These diapers, generally, comprise three basic components, a top sheet, an absorbent core and a bottom sheet, but may have additional components such as, but not limited to, an acquisition layer between the absorbent core and the top sheet. The acquisition layer may provide structure and features that cause wicking and capillary action to move liquids from the top sheet into the absorbent core.

Conventionally, absorbent cores of diapers comprise at least one of pulp, fluff pulp, hydrogels, and SAP polymers (superabsorbent polymers, for example, polyacrylate polymer) to absorb the liquids in urine and feces and keep the wearer's skin dry. By keeping the skin dry, these products help mitigate/prevent Incontinence Associated Dermatitis (IAD), an inflammation of the skin that occurs when urine or stool comes into contact with skin. Several skin care products are available, as well as different regimens are developed to help reduce the occurrence of IAD.

However, other problems may be exacerbated by the retention of urine and stool in a diaper and in an environment next to or in contact with the skin. Normally, the pH of healthy human skin is between 4.5 and 6.2, which is slightly acidic. Skin integrity becomes worse when the pH of the liquid coming into contact with the skin is alkaline. This is very much evident with older population groups and infants. The enzymes in feces (protease and lipase) may irritate the wearer's skin. In addition, feces may contain microorganisms that can cause skin infection in irritated areas. Urine further irritates the diaper area.

When urinary incontinence occurs for a person wearing a diaper, a significant portion of the liquid components of urine pass through the top sheet and are absorbed into the absorbent core. The liquid components of urine include, among other components, urea. Over time, the urea in the urine naturally degrades producing volatile compounds (carbon dioxide and ammonia). The degradation of urea may occur in the absorbent core of a diaper.

The urea degradation may be accelerated by naturally occurring enzymes. Urease is such a naturally occurring enzyme that catalyzes the degradation of urea. Most bacteria in skin flora, such as Staphylococcus, Corynebacterium and Micrococcus species have the urease enzyme and are capable of breaking down urea into ammonia and carbon dioxide. Unlike most enzymes that work inside the cell (intracellular), urease is an exoenzyme. An exoenzyme is an enzyme that operates outside the cell that produces it. Thus, urease is capable of breaking down urea present in the surroundings/environment around the skin flora. The presence of bacteria or bacterial urease in the absorbent core of a diaper results in the acceleration of degradation of urea producing carbon dioxide and ammonia.

As urine breaks down the ammonia that is released raises the pH of the surrounding environment to produce an alkaline environment within the diaper and next to the wearer's skin. The enzymes and microorganisms from the feces may become more active in this alkaline environment. This combination of actions exacerbates tissue/skin damage and leads to diaper rash and other irritations.

There exists a need for an incontinence device that reduces the degradation of urea within the diaper, therefore reducing odor and change in pH. There also exists a need for a diaper that reduces microbial activity in the diaper and/or on the wearer's skin.

### SUMMARY

Devices used to absorb bodily fluids include, but are not limited to, diapers, incontinence devices, feminine hygiene products, or wound dressings, for example. Embodiments of these devices comprise a top sheet, an absorbent core, and back sheet. Embodiments of the top sheet comprise a nonwoven polymeric sheet having urease inhibiting compounds and/or copper compounds. For example, the nonwoven polymeric top sheet comprises polymeric fibers and the polymeric fibers comprises a plurality of first particles of water insoluble copper compounds that release at least one of Cu+ ions and Cu++ ions upon contact with a body fluid embedded in at least a portion of the polymeric fibers and water soluble copper compounds on the surface of the polymeric fibers. The water soluble copper compounds on the surface are present may be present in an amount between 0.1 wt. % and 1.0 wt. % of the total weight of the top sheet. The insoluble copper compounds are embedded and protruding from the surface of the polymeric fibers and may be present in a concentration of water insoluble copper compounds embedded in the top sheet are between 0.1 wt. % and 1.0 wt. % of the total weight of the top sheet.

In one embodiment of the device, the top sheet may comprise polyethylene fibers having between 1.0 and 5.0 wt. % of one or more of cuprous oxide and cupric oxide particles embedded in the fibers and 100 and 500 ppm of one or more of copper sulfate particles on the surface of the fibers based upon the weight of the top sheet. The polypropylene fibers may be between 15 to 20 microns in diameter and comprise particles of water insoluble copper compounds with 90% of the particles having a particle size between 1 to 2 microns.

Further, embodiments of the absorbent core also comprise urease inhibiting compounds. In certain embodiments, the absorbent core comprises a body fluid absorbent material and a plurality of second particles of water soluble copper compound that release at least one of Cu+ ions and Cu++ ions upon contact with a body fluid embedded in the body fluid absorbent material.

The device for retaining a bodily fluid may further comprise a water insoluble back sheet, wherein the absorbent core is between the top sheet and the back sheet.

In another embodiment of the device for retaining body fluids may comprise a top sheet comprising a nonwoven polymeric sheet, wherein the nonwoven polymeric sheet comprises water insoluble urease inhibiting particles embedded within the polymeric sheet and water soluble urease inhibiting particles on the surface of the nonwoven polymeric sheet, an absorbent core comprising a water absorbent material and a water insoluble urease inhibiting particles within the super absorbent polymer, and a back sheet.

An embodiment of the method of forming a diaper, incontinence device, feminine hygiene product, or wound dressing that inhibits the catalytic hydrolysis of urea by urease may comprise adding water insoluble copper compounds that release at least one of Cu+ ions and Cu++ ions upon contact with a fluid into a process for the formation of the absorbent core, forming the nonwoven top sheet from polymeric fibers comprising water insoluble copper compounds and water soluble copper compounds that release at least one of Cu+ ions and Cu++ ions upon contact with a fluid and layering the nonwoven top sheet on one side of the absorbent core; and sealing the absorbent core on the other side with a water impermeable bottom sheet. International Application PCT/US22/39659 is incorporated by reference in its entirety.

### BRIEF DESCRIPTION OF THE DRAWINGS

FIG. 1. Is a depiction of the test apparatus to measure ammonia gas that passes through a top sheet with and without urease inhibitors present in the top sheet; and

FIG. 2 shows the layers of the device to retain body fluids 20 including the top sheet 21, the acquisition layer 22, the absorbent core 23, and the back sheet 24.

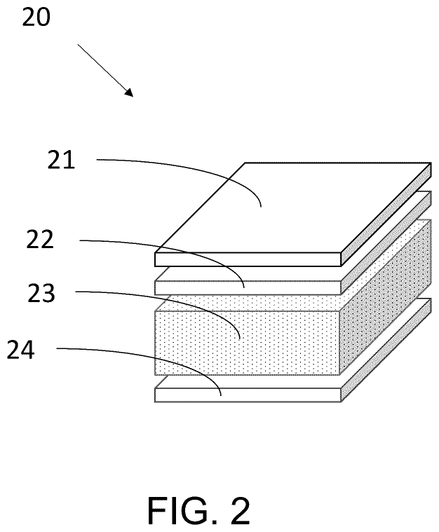

### DESCRIPTION

Incontinence devices retain urine and feces between changes. Sometimes the used incontinence device may be changed soon after use and other times there may be a significant delay between the evacuation of feces and urine and the removal and replacement of the incontinence device. During this period of time that the urine resides in the incontinence device, the urea may degrade to ammonia and carbon dioxide. These compounds are highly volatile and may evaporate quickly. Carbon dioxide is odorless. However, ammonia has an unpleasant odor to humans and a low odor threshold.

The inventors, surprisingly, have found that the ammonia formed during this process is not completely volatile and all does not escape into the environment even though ammonia is considered highly volatile. Significant amounts of ammonia, produced due to decomposition of urea in the urine, is absorbed by the other liquid components of the urea thereby raising the pH of the liquid.

A synthetic urine was developed to understand the chemical process of urea degradation in urine. The pH of synthetic urine in these experiments was initially acidic (pH=6.0) but, in the presence of urease, became alkaline (pH=8.0) over an 8.0-hour period. Upon addition of insoluble or soluble copper to the urine with bacteria/urease, the insoluble or soluble copper acts as a urease inhibitor. It is known that copper oxide is an antimicrobial but it was also found, surprisingly, to be urease inhibitor and prevented the urease catalyzed degradation of urine.

The top sheet of a diaper is typically a nonwoven polymeric material which may comprise a combination of pores and micro pores to allow liquid deposited on one side of the top sheet to transfer through the top sheet to the absorbent core of the diaper. Urine passes through and contacts the top sheet of the incontinence device to be absorbed and retained by the absorbent core. Nonwoven materials may be formed from polymeric materials comprising a concentration of particles of water insoluble copper compounds that release at least one of Cu+ ions and Cu++ ions upon contact with a fluid. The insoluble copper compounds are embedded and/or protruding from the surface of the polymeric materials and may exhibit antimicrobial, antiviral and/or antifungal properties. In an incontinence device top sheet, the insoluble copper compounds contact the skin of the wearer and may release copper ions into the sweat and/or urine of the wearer.

Wound dressings similarly comprise a top sheet for contacting the wound and an absorbent core for retaining wound exudate. Also, feminine hygiene products may also comprise a top sheet that contacts the skin and the absorbent core for retaining blood. These products may also benefit from the inventions described herein.

### Top Sheets

A top sheet may be a component of a device for retaining bodily fluids such as, but not limited to, an incontinence device, a feminine hygiene device, and a wound dressing, for example. A top sheet is typically placed in contact with the skin of the wearer when the device is in use. Thus, use of copper compounds in nonwovens incorporated in such products as top sheets inhibit formation of the ammonia by inhibiting the degradation of urea into ammonia and prevent the formed ammonia from reaching the skin effectively minimizing the chance of an increase in skin pH.

The copper compounds may be incorporated in the top sheet by embedding copper containing particles into the polymer fibers of the nonwoven or by applying the particle to the surface of the nonwoven. For example, a solution of soluble copper compound may be sprayed on the nonwoven, then dried to precipitate the particles on the fibers of the nonwoven.

For example, an embodiment of an incontinence device may comprise a top sheet comprising a nonwoven material formed from polymeric materials comprising a concentration of particles of water insoluble and/or soluble copper compounds that release at least one of Cu+ ions and Cu++ ions upon contact with a fluid. The incontinence device may further comprise an absorbent core, and a liquid impermeable bottom sheet. Further, upon exposure to urine, the copper oxide impregnated nonwovens inhibited urease degradation of the urea, prevented microbial growth on the skin, and blocked the release of ammonia from the absorbent core.

The concentration of water insoluble copper compounds (or other compounds as described herein) may be between 0.01 wt. % and 10 wt. % based on the weight of the top sheet. In other embodiments, the concentration of water insoluble copper compounds may be between 0.01 wt. % and 1.0 wt. % of the total weight of the top sheet. In another embodiment, the water insoluble copper compounds may be between 100 ppm and 5000 ppm based on the weight of the top sheet.

As stated, the top sheet may further be treated with water soluble copper compounds. The top sheet may be sprayed with a solution of soluble copper compounds. The soluble copper compounds may include, but not limited to, copper sulfate, for example. In an embodiment, the concentration of water soluble copper compounds applied to the surface may be between 0.01 wt. % and 1.0 wt. % of the total weight of the top sheet. In another embodiment, the water-soluble copper compounds may be between 100 ppm and 5000 ppm based on the weight of the top sheet. In a still further embodiment, the water soluble copper compounds may be between 50 ppm and 1000 ppm based on the weight of the top sheet.

In one embodiment, the top sheet may include water insoluble copper compounds embedded in the fiber and water-soluble copper compounds applied to the surface of the fibers. The top sheet may comprise polyethylene fibers having between 1.0 and 5.0 wt. % of one or more of cuprous oxide and cupric oxide particles embedded in the fibers and 100 and 500 ppm of one or more of copper sulfate particles on the surface of the fibers. The water-soluble copper compounds may be more readily available to the environment surrounding the device while the embedded copper compounds are more durable and slower to release of ions and may contact the skin.

The concentration and the particle size of the insoluble copper compounds embedded in the nonwoven fabric and/or water-soluble copper compounds applied to the surface of the nonwoven may be limited by factors including, but not limited to, the tactile feel of the nonwoven material on the skin of the wearer, the tensile strength of the nonwoven, and the processing efficiency of the nonwoven material, for example. For example, a top sheet may comprise polypropylene fibers having 500 ppm of one or more of cuprous oxide and cupric oxide particles embedded in the fibers. At least a portion of the particles protruding from the surface of the polypropylene fibers. In one example of this embodiment the polypropylene fiber would be of 15 to 20 microns in diameter, treated with insoluble copper compounds of 1 to 2 microns in diameter.

### Absorbent Core

The absorbent core may additionally comprise water insoluble and/or soluble copper compounds that release at least one of Cu+ ions and Cu++ ions upon contact with a fluid. In embodiments, the absorbent core may comprise various components including, but not limited to, pulp, fluff pulp, cellulose fiber pulp, natural fibers, super absorbent polymer, staple fibers, synthetic fibers, or other hydrogels. Any component individually or a combination of components of the absorbent core of the device for retaining bodily fluids may be modified to include insoluble or water-soluble copper compounds that release at least one of Cu+ ions and Cu++ ions upon contact with a fluid such as, but not limited to, copper oxides or other beneficial particles as described herein. The absorbent core comprising the copper compounds would absorb the urea or other bodily exudate and the associated urease, microbes, and/or other enzymes carried from the skin of the wearer.

In the absorbent core of an incontinence device, the copper compounds inhibit the catalytic action of the hydrolysis of urease thus substantially inhibiting the formation of carbon dioxide and ammonia. Thus, maintaining the acidity of the urine and preventing formation of an alkaline environment that results in irritation of the skin.

Thus, a further embodiment of a diaper comprises a top sheet comprising a nonwoven material, an absorbent core formed of an absorbent material comprising a concentration of particles of water insoluble copper compounds that release at least one of Cu+ ions and Cu++ ions upon contact with a fluid, and a liquid impermeable bottom sheet.

A still further embodiment of a diaper comprises a top sheet comprising a nonwoven material formed from polymeric materials comprising a concentration of particles of water insoluble copper compounds that release at least one of Cu+ ions and Cu++ ions upon contact with a fluid, an absorbent core formed of an absorbent material comprising a concentration of particles of water insoluble copper compounds that release at least one of Cu+ ions and Cu++ ions upon contact with a fluid, and a liquid impermeable bottom sheet.

Employing the described copper compound along with SAP & pulp in the absorbent core and/or the top sheet would reduce the formation of alkaline environment on the skin.

### Antimicrobial and Urease inhibitors

The invention has been primarily exemplified with copper particles, however, the particles may be any particles which provide desired properties to the fiber including, but not limited to, cuprous oxide (Cu₂O), cupric oxide (CuO), cuprous iodide (CuI), cuprous thiocyanate (CuSCN), copper sulfide, copper sulfate (CuSO₄), copper chloride (CuCl₂), zeolites, especially copper-zeolites, zirconium phosphate, copper-zirconium phosphate, zinc pyrithione, zinc oxide, titanium oxide, titanium dioxide, titanium oxide, silver nitrate, silver oxide, and silver oxide, silver iodide, silver chloride, silver sulfate, silver sulfide, quaternary ammonium compounds, and combinations thereof. In the description of specific examples, any of the listed particles may be substituted to provide the desired static or dynamic properties.

### Definitions

1. Incontinence Associated Dermatitis (IAD): An inflammation of the skin that occurs when urine or stool comes into contact with perineal or perigenital skin.

2. Skin's acid mantle: The acid mantle is a very fine, slightly acidic film on the surface of human skin acting as a barrier to bacteria, viruses and other potential contaminants that might penetrate the skin. It is secreted by sebaceous glands. The pH of the skin differs between people and conditions and may be between 4.5 and 6.2, slightly acidic.

3. Urine: A liquid by-product of the body secreted by the kidneys through a process called urination (or micturition) and excreted through the urethra. The composition of urine varies from person to person and is dependent of dietary and health conditions etc. However, the main ingredients in urine are water, sodium chloride (NaCl) and urea (NH2—CO—NH2) and below is list of major constituents and corresponding approximate concentrations of each in water:
5a. Inorganic salts:
I. Sodium Chloride: 8,001 mg/l
II. Potassium sulfate: 2,632 mg/l
III. Potassium Chloride: 1,641 mg/l
IV. Magnesium sulfate: 783 mg/l
10b. Organic compounds:
I. Urea: 13,400 mg/l
II. Creatinine: 1,504 mg/l
III. Ammonium Hippurate: 1,250 mg/l

4. Urease: Urease is an exoenzyme that catalyzes the hydrolysis of urea into carbon dioxide and ammonia as shown by the equation below:

NH2—CO—NH2+H2O [symbol] 4NH3↑+2CO2↑

5. Urease inhibitors: Chemicals or agents that slow or inhibit the above reaction. These are broadly classified into two classes: a) Substrate structure analogs (similar to Urea in structure); b) Inhibitors that affect the mechanism. Some heavy metal ions, such as lead (Pb+2), mercury (Hg+2) including Cupric ions (Cu+2) are known to inhibit or slow down the activity of Urease.

### Methods for the Addition of Cupron to Absorbent Core of Diaper

During the manufacture of a incontinence device, a feminine hygiene product or a wound dressing, the absorbent core may be formed on a moving conveyer belt. This step of the process may be referred to as the “forming chamber.” The fiber pulp is first defibrized using a hammer mill. At different points in this chamber, SAP (superabsorbent polymer) particles or fluff pulp (fibrous material) are sprayed onto the conveyor belt using pressurized nozzles. The bottom of the conveyor is perforated and vacuum is applied from below the conveyor belt to form a flat pad from the material sprayed onto the belt.

There are couple of different ways to incorporate absorbent polymers into the absorbent core. The polymer can be injected directly into pulp. This method helps with even distribution of absorbent polymer throughout the pad. Another way is to apply polymer over the pulp fiber i.e., the absorbent material is placed onto the top surface of the pad after it has been formed. Alternatively, multiple spray dispensers can be used to apply several layers of fiber, polymer and fiber as a sandwich. This creates a pad with the absorbent polymer in the center with pulp material on the outside.

The water-soluble and/or water insoluble particles may be added to the absorbent core during this process, for example. The different ways to incorporate the urease inhibiting insoluble copper compounds into the absorbent core include, but are not limited to:
1. The urease inhibiting insoluble copper compounds and/or a solution comprising water-soluble copper compounds could be added through a separate pressure nozzle during the formation of the absorbent core.
2. The urease inhibiting insoluble copper compounds and/or a solution comprising water-soluble copper compounds could be added to the SAP particles and dry blended at different ratios and sprayed together on to the conveyor belt on to the pulp.
3. The urease inhibiting insoluble copper compounds and/or a solution comprising water-soluble copper compounds could be added to the pulp during the defibrizing process to the hammer mill.
4. The urease inhibiting insoluble copper compounds and/or a solution comprising water-soluble copper compounds, SAP and defibrized pulp can be pre-blended together and sprayed onto the conveyor belt through one nozzle.
5. Small amounts of water are sprayed on to the pulp-SAP layers on the conveyor belt to prevent static discharge. The urease inhibiting insoluble copper compounds and/or a solution comprising water-soluble copper compounds could be added to the absorbent core at this time through copper containing solutions.
6. Staple fibers comprising embedded copper compounds may be added and distributed evenly to the superabsorbent polymer.

In another embodiment, a method of forming a diaper that inhibits the catalytic hydrolysis of urea by urease comprises adding water insoluble and/or water-soluble copper compounds that release at least one of Cu+ ions and Cu++ ions upon contact with a fluid into a process for the formation of the absorbent core; layering a nonwoven top sheet on one side of the absorbent core; and sealing the absorbent core on the other side with a water impermeable bottom sheet.

Another embodiment of the method may include forming the nonwoven top sheet of an incontinence device, feminine hygiene product, or wound dressing, for example, from a polymeric material comprising water insoluble copper compounds and/or water-soluble copper compounds that release at least one of Cu+ ions and Cu++ ions upon contact with a fluid. Such a top sheet may be combined with the absorbent core to provide a symbiotic product that prevents urea degradation, odor formation, and reduces growth of irritating microbes on the skin.

### Bottom Sheet

In a still further embodiment of the method may include forming the bottom sheet of the diaper from a sheet of a polymeric material comprising water insoluble copper compounds that release at least one of Cu+ ions and Cu++ ions upon contact with a fluid.

In certain embodiments, the antimicrobial copper compounds may be added to a melted polymer to form polymeric slurry. The polymers may include, but are not limited to, a polyester, polyolefins, polyethylene, high density polyethylene, low density polyethylene, polystyrene, polyacrylates, polymethacrylates, polyethylene terephthalate (PET), polybutylene terephthalate (PBT), polytrimethylene terephthalate, polylactic acid (PLA), polyglycolide (PGA), polylactic-co-glycolic acid (PLGA), polyamides or nylon including, but not limited to, nylon-6 (polycaprolactum) and Nylon 66, polyurethanes, similar thermoplastic polymers or copolymers, super absorbent polymers, and combinations thereof. Polyesters are polymers formed from a dicarboxylic acid and a diol. The polymer may be extruded to produce fibers, yarns or sheets which possess antimicrobial, antifungal and/or antiviral properties.

### EXAMPLES

A series of experiments were conducted to determine and demonstrate the efficacy of water soluble and insoluble copper compounds in different components of diapers that release at least one of Cu+ ions and Cu++ ions upon contact with a fluid to mitigate the pH increase due to metabolism of urea, a major constituent of urine, by urease and urease producing bacteria.

Typically, human urine contains ˜13.4 grams/liter of urea, a major constituent, along with other inorganic salts and organic constituents. The degradation of urea in the urine may be catalyzed by urease-containing bacteria into ammonia and carbon dioxide according to the following reaction:

NH₂—CO—NH₂→2NH₃+CO₂

The carbon dioxide formed during the process escapes into the environment as a gas. However, the ammonia can absorb into the Urine solution or sweat on the skin to form of ammonium hydroxide (NH₄OH) and thus raise the pH of Urine or microenvironment of the skin.

### Example 1

### Experimental Set Up and Background

A synthetic urine composition was produced and used in the experiments from (1-4). The following components were dissolved in water as follows:
1. Urea: 13,400 mg/l
2. Sodium Chloride: 8,500 mg/l
3. Sodium sulfate: 1,000 mg/l
4. 5.0% NB (25 ml of DIFCO Nutrient Broth solution (8.0 grams/liter) per 500 ml of solution)

### Experiment Setup—1

Synthetic urine is prepared, in bulk, using the composition described above. Six specimen cups, 120 ml capacity, with 50 ml of synthetic urine solution in each are taken. Two cups served as complete blanks, one without any fabric impregnated with copper compounds or bacteria and the other without bacteria but with fabric impregnated with copper compounds. 100 μl of Klebsiella pneumonia (urease producing bacteria, 10⁸ CFU/ml) solution was added to rest of the four cups and 0.5 grams of fabric impregnated with copper compounds was added to only two of the four cups as shown in the table below. pH of the solutions was measured using pH indicator strips. The pH of the solutions without any fabric impregnated with copper compounds raised from 6.0 to 8.0 in 8 hours indicating the ammonia formation due to urea metabolism while the pH of the solutions with fabric impregnated with copper compounds stayed at 6.0 indicating the inhibition of urease enzyme and bacteria producing urease enzyme.

| | | Polyester | | | |
|---|---|---|---|---|---|
| Sl. | Staphylococcus | fabric impregnated with 1.0% | pH @ 0 | pH after | |
| No. | epidermis | Cuprous Oxide | hour | 8 hours | Δ pH |
| B1 | 0 cfu | None | 6.0 | 6.0 | 0.0 |
| B2 | | 0.5 grams | 6.0 | 6.0 | 0.0 |
| B3 | 10⁶ cfu | None | 6.0 | 8.0 | 2.0 |
| B4 | | None | 6.0 | 8.0 | 2.0 |
| B5 | | 0.5 grams | 6.0 | 6.0 | 0.0 |
| B6 | | 0.5 grams | 6.0 | 6.0 | 0.0 |

### Example 2

Similar experiment as above were run but this time, the polypropylene fabric embedded with 3% copper compounds was replaced with polyester fabric embedded with 1% copper compounds in the synthetic urine. Similar results, as in Example 1, were observed.

| | | Polypropylene | | | |
|---|---|---|---|---|---|
| Sl. | Klebsiella | Non-woven fabric impregnated | pH @ 0 | pH after | |
| No. | pneumonia | with 3.0% Cuprous Oxide | hour | 8 hours | Δ pH |
| A1 | 0 cfu | None | 6.0 | 6.0 | 0.0 |
| A2 | | 0.5 grams | 6.0 | 6.0 | 0.0 |
| A3 | 10⁶ cfu | None | 6.0 | 8.0 | 2.0 |
| A4 | | None | 6.0 | 8.0 | 2.0 |
| A5 | | 0.5 grams | 6.0 | 6.0 | 0.0 |
| A6 | | 0.5 grams | 6.0 | 6.0 | 0.0 |

### Example 3

### Experiment Setup—2

Synthetic urine was again prepared, in bulk, using the composition described above. Six petri dishes, 90 mm size, were taken and 1.0 gram of diaper absorbent core material (fluff with SAP) was added to each. Synthetic urine sample, with or without Cuprous oxide as shown in the table below, was added to each petri dish until the diaper material was complexly saturated. Samples C3, C4, C5 and C6 were inoculated with 100 μl of urease solution. pH indicator strips were pressed into the material to measure the pH. The experiments show that the addition of copper oxide to the diaper absorbent core prevents the action of Urease enzyme and change in 10 pH of the urine.

| Sl. | | Cuprous oxide in | pH @ 0 | pH after | |
|---|---|---|---|---|---|
| No. | Urease | 100 ml of Synthetic Urine | hour | 2 hours | Δ pH |
| C1 | No | None | 6.0 | 6.0 | 0.0 |
| C2 | | 0.05 grams | 6.0 | 6.0 | 0.0 |
| C3 | Yes | None | 6.0 | 8.0 | 2.0 |
| C4 | | None | 6.0 | 8.0 | 2.0 |
| C5 | | 0.05 grams | 6.0 | 6.0 | 0.0 |
| C6 | | 0.05 grams | 6.0 | 6.0 | 0.0 |

### Example 4

### Experiment Setup—3

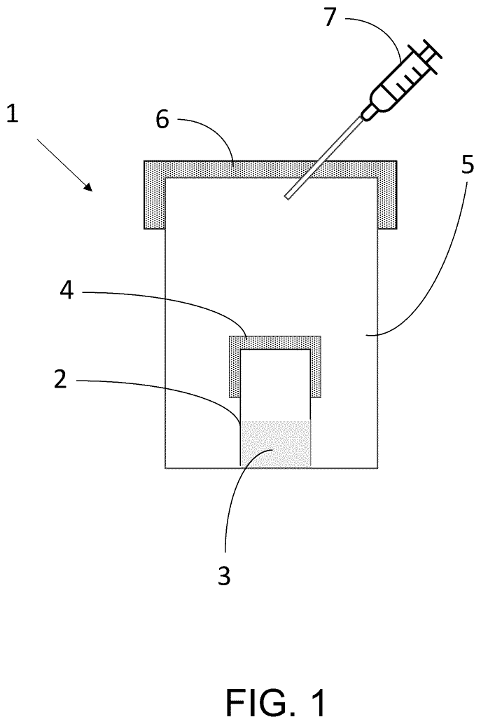

In a different set up as shown in the FIG. 1, four 20 milliliter glass vials 2 with 10 ml of urine solution 3, in each vial, are prepared. All four vials were inoculated with 10 μl of urease enzyme. Two of the vials (control) were covered tightly with regular nonwoven fabric 4 (without any copper compounds) and the other two were covered with 3% cuprous oxide containing polypropylene nonwoven fabric 4. Further, all vials sealed with fabric, were placed in 120 ml specimen container 5 that is sealed airtight with parafilm 6. Ammonia concentrations were obtained using Sensidyne pump (Model: AP-20S Gas Detection Pump) and ammonia gas detection tubes 7. The 3% cuprous oxide containing polypropylene nonwoven fabric were able to capture and minimize the amount of ammonia escaped into the environment significantly. The results are shown in the table below.

| | | | Ammonia, in | Ammonia, in | |
|---|---|---|---|---|---|
| Sl. | | polypropylene | ppm, @ 0 | ppm, after 8 | Δ Ammonia, |
| No. | Urease | Non-woven fabric | hour | hours | in ppm |
| D1 | YES | Regular nonwoven | 0.0 | 7.0 | 7.0 |
| | | fabric | | | |
| D2 | | Regular nonwoven | 0.0 | 6.5 | 6.5 |
| | | fabric | | | |
| D3 | | Cupron nonwoven | 0.0 | 2.0 | 2.0 |
| | | fabric with 3% copper | | | |
| | | oxide | | | |
| D4 | | Cupron nonwoven | 0.0 | 2.5 | 2.5 |
| | | fabric with 3% copper | | | |
| | | oxide | | | |

### Examples (5-11)

A slightly different synthetic urine composition was prepared and used in the following experiments from (5-11). The following components were dissolved in deionized water as follows:
1. Urea: 20,000 mg/l
2. Phenol red (a pH indicator): 20 mg/l
3. Sodium Chloride: 800 mg/l, Sodium phosphate: 1420 mg/l, Potassium phosphate: 240 mg/l 104. 0.5% TSB (tryptic soy broth solution)
5. 0.05% triton x-100

### Example 5: Top Sheet with Soluble Copper on Top of a Regular Cellulose Core (w/o any Additive), Incubated with Proteus mirabilis for 13 Hours

A 25 gsm polypropylene nonwoven Top sheet was treated with varying amounts of copper sulfate solutions and dried overnight. The treated Top sheet is placed on top of a core made of cellulose material. To this system (comprising Top sheet and the cellulose core), synthetic urine solution containing Proteus mirabilis is added and incubated for 13 hours at 37±2° C.

| | Soluble | | | | |
|---|---|---|---|---|---|
| | Copper | Concentration of | | | |
| | concentration | Copper in the system | | | |
| Sl. | on Top sheet | (top sheet + cellulose | pH | pH | |
| No. | (ppm) | core) (ppm) | (time = 0 hr) | (time = 13 hr) | ΔpH |
| 1 | 0 | 0 | 6 | 9 | 3.0 |
| 2 | 5 | 0.5 | 6 | 9 | 3.0 |
| 3 | 50 | 5 | 6 | 9 | 3.0 |
| 4 | 500 | 50 | 6 | 9 | 3.0 |
| 5 | 5000 | 500 | 6 | 6 | 0.0 |

### Example 6: Regular Top Sheet (w/o Additive) on Top of a Cellulose Core Treated with Soluble Copper, Incubated with Proteus mirabilis for 13 Hours.

A cellulose core was treated with varying amounts of copper sulfate solutions and dried overnight. Untreated Top sheet is placed on top of the cellulose core treated with varying amount of copper sulfate. To this system (comprising Top sheet and treated cellulose core), synthetic urine solution containing Proteus mirabilis is added and incubated for 13 hours at 37±2° C.

| | | Concentration of | | | |
|---|---|---|---|---|---|
| | Soluble Copper | Copper in the system | | | |
| Sl. | concentration | (top sheet + cellulose | pH | pH | |
| No. | in Core (ppm) | core) (ppm) | (time = 0 hr) | (time = 13 hr) | ΔpH |
| 1 | 0 | 0 | 6 | 9 | 3.0 |
| 2 | 5 | 5 | 6 | 9 | 3.0 |
| 3 | 50 | 50 | 6 | 7 | 1.0 |
| 4 | 500 | 500 | 6 | 6 | 0.0 |
| 5 | 5000 | 5000 | 6 | 6 | 0.0 |

### Example 7: Both Top Sheet and Cellulose Core Treated with Soluble Copper, Incubated with Proteus mirabilis for 13 Hours

A 25 gsm polypropylene nonwoven Top sheet was treated with varying amounts of copper sulfate solutions and dried overnight. A cellulose core was treated with varying amounts of copper sulfate solutions and dried overnight. Top sheet is placed on top of the cellulose core and to this system (comprising Top sheet and treated cellulose core), synthetic urine solution containing Proteus mirabilis is added and incubated for 13 hours at 37±2° C.

| | | Soluble | Concentration of | | | |
|---|---|---|---|---|---|---|
| | Soluble | Copper | Copper in the | | | |
| | Copper | concentration | system (top | | | |
| | concentration | in Core (as | sheet + cellulose | pH | ph | |
| SI. | on Top sheet | CuSO4, in | core) (as CuSO4, | (time = | (time = | |
| No. | (ppm) | ppm) | in ppm) | 0 hr) | 13 hr) | ΔpH |
| 1 | 0 | 0 | 0 | 6 | 9 | 3.0 |
| 2 | 0 | 50 | 50 | 6 | 7 | 1.0 |
| 3 | 500 | 0 | 50 | 6 | 9 | 3.0 |
| 4 | 50 | 50 | 55 | 6 | 6 | 0.0 |

### Example 8: Top Sheet Treated with Soluble Copper on Top of Regular SAP Core (Super Absorbent Polymer Core, w/o any Additive), Incubated with Proteus mirabilis for 13 Hours

A 25 gsm polypropylene nonwoven Top sheet was treated with varying amounts of copper sulfate solutions and dried overnight. The treated Top sheet is placed on top of the core made of SAP (superabsorbent polymer made with polyacrylate polymer) material. To this system (comprising Top sheet and the SAP core), synthetic urine solution containing Proteus mirabilis is added and incubated for 13 hours at 37±2° C.

| | | Final concentration of | | |
|---|---|---|---|---|
| | Soluble Copper | Copper in the system | | |
| | concentration on | (top sheet+cellulose | | |
| Sl. | Top sheet (as | core) (as CuSO4, in | pH | pH |
| No. | CuSO4, in ppm) | ppm) | (time = 0 hr) | (time = 13 hr) |
| 1 | 0 | 0 | 6 | 8 |
| 2 | 3000 | 83.3 | 6 | 8 |
| 3 | 5000 | 138.9 | 6 | 8 |

### Example 9A: Both Top Sheet and Diaper Core Treated with Soluble Copper, Incubated with Proteus mirabilis for 3 & 22 Hours

A 25 gsm polypropylene nonwoven Top sheet was treated with varying amounts of copper sulfate solutions and dried overnight. The treated Top sheet is placed on top of the diaper core made of cellulose and SAP (superabsorbent polymer made with polyacrylate polymer) material. To this system (comprising Top sheet and the diaper core), synthetic urine solution containing Proteus mirabilis is added and incubated for 13 hours at 37±2° C. pH readings were obtained using pH sticks at 0, 3 and 22 hour incubation periods and recorded as below:

| | Soluble | | | | | |
|---|---|---|---|---|---|---|
| | Copper | Soluble | Concentration of | | | |
| | concentration | Copper | Copper in the | | | |
| | on Top | concentration | system (top | | | |
| | sheet (as | in Core (as | sheet + cellulose | pH | pH | pH |
| SI. | CuSO4, in | CuSO4, in | core) (as CuSO4, | (time = | (time = | (time = |
| No. | ppm) | ppm) | in ppm) | 0 hr) | 3 hr) | 22 hr) |
| 1 | 0 | 0 | 0 | 6 | 9.5 | 10 |
| 2 | 0 | 71.4 | 71.4 | 6 | 8 | >9.0 |
| 3 | 0 | 142.8 | 142.8 | 6 | 7.5 | >9.0 |
| 4 | 50,000 | 0 | 700 | 6 | 6 | 7.5 |
| 4 | 100,000 | 0 | 1400 | 6 | 6 | 6.5 |

### Example 9B: Both Top Sheet and Diaper Core Treated with Soluble Copper, Incubated with Proteus mirabilis for 3 & 22 Hours

A 25 gsm polypropylene nonwoven Top sheet was treated with varying amounts of copper sulfate solutions and dried overnight. The treated Top sheet is placed on top of the diaper core made of cellulose and SAP (superabsorbent polymer made with polyacrylate polymer) material. To this system (comprising Top sheet and the diaper core), synthetic urine solution containing Proteus mirabilis is added and incubated for 13 hours at 37±2° C. Odor readings were obtained using Sensidyne ammonia gas detector tubes at 0, 3 and 22 hour incubation periods. The odor readings were obtained in similar fashion as described in FIG. 1 and experimental set-up 3, and recorded as below:

| | Soluble | | Concentration of | | |
|---|---|---|---|---|---|
| | Copper | Soluble Copper | Copper in the | | |
| | concentration | (as CuSO₄) | system (top | Odor, as | Odor, as |
| Sl. | on Top sheet | concentration | sheet + cellulose | ammonia (@ | ammonia (@ |
| No. | (ppm) | in Core (ppm) | core) (ppm) | time = 0 hr) | time = 3 hr) |
| 1 | 0 | 0 | 0 | 0 | >100 |
| 2 | 0 | 71.4 | 71.4 | 0 | 100 |
| 3 | 0 | 142.8 | 142.8 | 0 | 50 |
| 4 | 50,000 | 0 | 714.3 | 0 | 15 |
| 4 | 100,000 | 0 | 1428.6 | 0 | 0 |

### Example 10: Urease Enzyme Activity and Corresponding Change in pH in Urea Containing Solution with Varying Copper Concentrations

| | Copper (as CuSO₄) | | | |
|---|---|---|---|---|
| Sl. | concentration in solution | pH | pH | |
| No. | (ppm) | (time = 0 hr) | (time = 10 min) | ΔpH |
| 1 | 0 | 6.0 | 9.0 | 3.0 |
| 2 | 5 | 6.0 | 7.0 | 1.0 |
| 3 | 50 | 6.0 | 6.0 | 0.0 |
| 4 | 500 | 5.5 | 5.5 | 0.0 |
| 5 | 5,000 | 5.0 | 5.0 | 0.0 |
| 6 | 50,000 | 5.0 | 5.0 | 0.0 |

### Example 11: Effect of Soluble Copper Concentration on SAP Absorbency

Copper ions, at higher concentrations, have demonstrated to adversely impact the absorbency of the SAP polymer.

| | | Liquid | |
|---|---|---|---|
| | | absorbed per | % Reduction |
| Sl. | Copper concentration | 0.1 grams of | in |
| No. | in solution (ppm) | SAP, ml | absorbency |
| 1 | 0 | 8.0 | N/A |
| 2 | 5 | 8.0 | 0.0% |
| 3 | 50 | 8.0 | 0.0% |
| 4 | 500 | 8.0 | 0.0% |
| 5 | 5,000 | 5.5 | 31.3% |
| 6 | 50,000 | 2.0 | 75.0% |

### Example 12

An embodiment of a diaper comprises a top sheet comprising a nonwoven material formed from polymeric materials comprising a concentration of particles of water insoluble copper compounds that release at least one of Cu+ ions and Cu++ ions upon contact with a fluid, an absorbent core, water soluble copper compounds that release at least one of Cu+ ions and Cu++ ions upon contact with a fluid, and a liquid impermeable bottom sheet. The concentration of water insoluble copper compounds may be between 0.05 wt. % and 1.0 wt. % based on the weight of the polymeric material. In another embodiment, the water insoluble copper compounds may be between 1 wt. % and 4 wt. % based on the weight of the polymeric material.

### Example 13

An embodiment of an incontinence device may be prepared comprising a top sheet comprising a nonwoven material formed from polymeric materials comprising a concentration of particles of water insoluble copper compounds that release at least one of Cu+ ions and Cu++ ions upon contact with a fluid, wherein the top sheet comprises 3 wt. % of the insoluble copper compounds embedded in a polypropylene nonwoven. The absorbent core comprises a mixture of super absorbent polymer and cellulose fiber pulp. The cellulose fiber pulp is sprayed with copper sulfate solution such that the dried absorbent core comprises 2wt. % of water soluble copper compounds that release at least one of Cu+ ions and Cu++ ions upon contact with a fluid. The diaper also had a liquid impermeable bottom sheet.

### Example 14

An embodiment of an incontinence device may be prepared comprising a top sheet comprising a nonwoven material formed from polymeric materials comprising a concentration of particles of water insoluble copper compounds that release at least one of Cu+ ions and Cu++ ions upon contact with a fluid, wherein the top sheet comprises 0.05 wt. % (500 ppm) of the insoluble copper compounds embedded in a polypropylene nonwoven. The absorbent core comprises a mixture of super absorbent polymer and cellulose fiber pulp. The cellulose fiber pulp is sprayed with copper sulfate solution such that the dried absorbent core comprises 1.2 wt. % of water soluble copper compounds. The diaper also had a liquid impermeable bottom sheet.

The absorbent core will be formulated by spraying the cellulose fiber pulp with the copper sulfate solution and drying the pulp. The pulp may then be mixed with the super absorbent polymer to form the absorbent core.

1. A device for absorbing body fluids, comprising
a top sheet comprising a nonwoven polymeric sheet, wherein the nonwoven polymeric top sheet comprises polymeric fibers and wherein the polymeric fibers comprising a plurality of first particles of water insoluble copper compounds that release at least one of Cu+ ions and Cu++ ions upon contact with a body fluid embedded in at least a portion of the polymeric fibers and water soluble copper compounds on the surface of the polymeric fibers;
an absorbent core, wherein the absorbent core comprises a body fluid absorbent material and a plurality of second particles of water soluble copper compound that release at least one of Cu+ ions and Cu++ ions upon contact with a body fluid embedded in the body fluid absorbent material; and
a water insoluble back sheet, wherein the absorbent core is between the top sheet and the back sheet.

2. The device of claim 1, wherein the water soluble copper compounds on the surface are present in an amount between 0.1 wt. % and 1.0 wt. % of the total weight of the top sheet.

3. The device of claim 1, wherein the insoluble copper compounds are embedded and protruding from the surface of the polymeric fibers.

4. The device of claim 4, wherein the first particles of water insoluble copper compounds are one of copper oxides, copper iodides, and combinations thereof.

5. The device of claim 1, wherein the copper compounds comprise one of cuprous oxide and cupric oxide.

6. The device of claim 1, wherein the polymeric nonwoven material comprises polyethylene

7. The device of claim 1, wherein the body fluid absorbent material is a super absorbent polymer.

8. The device of claim 1, wherein the absorbent core comprises a concentration of water soluble copper compounds.

9. The device of claim 1, wherein the concentration of water insoluble copper compounds embedded in the top sheet are between 0.1 wt. % and 1.0 wt. % of the total weight of the top sheet.

10. The device of claim 1, wherein the top sheet comprises polyethylene fibers having between 1.0 and 5.0 wt. % of one or more of cuprous oxide and cupric oxide particles embedded in the fibers and 100 and 500 ppm of one or more of copper sulfate particles on the surface of the fibers based upon the weight of the top sheet.

11. The device of claim 10, wherein the polypropylene fibers are between 15 to 20 microns in diameter and comprise particles of water insoluble copper compounds with 90% of the particles having a particle size between 1 to 2 microns.

12. A device for retaining body fluids, comprising:
a top sheet comprising a nonwoven polymeric sheet, wherein the nonwoven polymeric sheet comprises water insoluble urease inhibiting particles embedded within the polymeric sheet and water soluble urease inhibiting particles on the surface of the nonwoven polymeric sheet;
an absorbent core comprising a water absorbent material and a water insoluble urease inhibiting particles within the super absorbent polymer; and
a back sheet.

13. A method of forming a diaper, of an incontinence device, feminine hygiene product, or wound dressing that inhibits the catalytic hydrolysis of urea by urease comprising:
adding water insoluble copper compounds that release at least one of Cu+ ions and Cu++ ions upon contact with a fluid into a process for the formation of the absorbent core;
forming the nonwoven top sheet from polymeric fibers comprising water insoluble copper compounds and water soluble copper compounds that release at least one of Cu+ ions and Cu++ ions upon contact with a fluid; and
layering the nonwoven top sheet on one side of the absorbent core; and sealing the absorbent core on the other side with a water impermeable bottom sheet.
